(19) United States
(12) Patent Application Publication
(10) Pub. No.: US 2006/0002982 A1
(43) Pub. Date: Jan. 5, 2006

(54) Xanthan gum viscoelastic composition, method of use and package

(75) Inventors: Joseph C. Salamone, Fairport, NY (US); Dharmendra M. Jani, Fairport, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/120,829

(22) Filed: May 3, 2005

Related U.S. Application Data
(60) Provisional application No. 60/584,096, filed on Jun. 30, 2004.

(51) Int. Cl. A61F 2/00

(52) U.S. Cl. 424/427

(57) ABSTRACT

The present invention is a novel viscoelastic composition that includes a suitable aqueous carrier and xanthan gum. The novel viscoelastic composition has rheological properties consistent with a good dispersive viscoelastic and improved damping ability over commercial dispersive viscoelastics.

### CROSS REFERENCE

This Application claims the benefit under 35 USC 119(e) of Provisional Patent Application No. 60/584,096 filed Jun. 30, 2004.

### BACKGROUND OF THE INVENTION

1. Field of the Invention

This invention relates to viscoelastic compositions, methods of use and related devices used in viscosurgical applications and more particularly to viscoelastic compositions, methods of use and related devices used in ophthalmic surgical application such as cataract removal surgery.

2. Discussion of Related Art

In the past decade, advances in the technology of eye surgery have made surgical treatment of eye disease and deformities attractive to alternative therapies. Cataract removal is one of the more common surgical procedures. Cataracts are opacities of the ocular lens, which generally arise in the elderly. Typically, cataract surgery involves removal of the cataractous lens from the capsular bag and replacement of the cataractous lens with a synthetic intraocular lens. Presently, this procedure involves making an incision through the sclera or cornea into the anterior chamber of the patient's eye. Another incision is made into the capsular bag. The cataractous lens is fractured in the capsular bag by procedures such as phacoemulsification and removed from the capsular bag by procedures such as aspiration. Thereafter, an intraocular lens is inserted into the capsular bag and deployed therein. The overall procedure is potentially traumatic to the tissue surrounding the anterior chamber. It is advantageous to reduce the amount of trauma to any living tissue in the patient eye during a surgical procedure. Particularly, corneal endothelial cells in the capsular bag are sensitive to damage, which is often irreversible. Serious damage can cause loss of eyesight and failure of the surgical procedure.

A viscoelastic composition is injected in the anterior chamber of the eye and the capsular bag during surgery to protect the tissue from physical trauma. The viscoelastic composition provides a physical barrier or cushion between the instruments and the tissue. Furthermore, the viscoelastic composition maintains the shape of a cavity during operation including the anterior chamber and capsular bag.

In addition to cataract surgery, viscoelastic compositions are useful in reducing tissue trauma and maintaining space of a cavity during other ophthalmic surgical procedures, including but not limited to trabeculectomy and vitrectomy.

Viscoelastic compositions have properties that make them effective for use in eye surgery to maintain the shape of a cavity and to protect the tissue. Viscoelastic compositions under zero-shear or low-shear preferably have relatively high viscosity. High viscosity compounds under zero-shear or low-shear conditions have better space maintenance properties than low viscosity compounds. However, it is difficult to inject or remove a highly viscous liquid through a cannula used for surgical procedures inside the eye. It is highly desirable to have a compound that has low viscosity under high-shear conditions and high viscosity under zero-shear or low-shear conditions. Generally, the ratio of the shear rate at a low-shear condition to a high-shear condition is the pseudoplasticity index. It is desirable for a viscoelastic composition to have high pseudoplasticity.

Common viscoelastic compositions for eye surgery include sodium hyaluronate (Healon® by Pfizer, New York, N.Y.), sodium hyaluronate and chondroitin sulfate (Viscoat® by Alcon Laboratories, Fort Worth, Tex.), hydroxypropylmethylcellulose (Ocucoat® by Bausch & Lomb, Rochester, N.Y.).

A composition whose viscoelastic component is essentially sodium hyaluronate has good shape maintaining characteristics, but is less effective at protecting the cells against damage during phacoemulsification.

A composition with hydroxypropylmethylcellulose and mixtures of hyaluronic acid and chondroitin sulfate are two viscoelastic compositions with dispersive viscoelastic properties. There is a need for a product that has a combination of both dispersive and cohesive properties.

Xanthan Gum is a hetero-polysaccharide of high molecular weight. Its main chain comprises glucose units. The side chain is a trisaccharide, consisting of alpha-D-mannose with an acetyl group, beta-D-glucuronic acid, and a terminal beta-D-mannose unit linked with a pyruvate group. The structure is represented by the following:

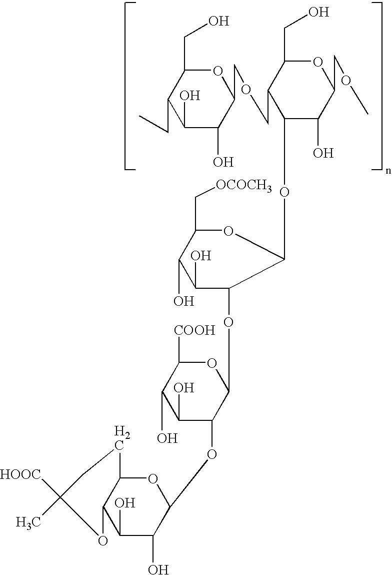

Xanthan Gum is produced as a secondary metabolite by a biotechnological fermentation process, based on the culture Xanthomonas campestris under aerobic conditions.

Xanathan Gum has been used as a congealing agent in pastry fillings, sauces and gravies, pourable salad dressings and in dairy products. However, food products do not require the level of purity of a viscosurgical device.

Oviatt, et al, Int. J. Biol. Macro., vol. 15, (February 1993) describes a method of purifying xanthan gum.

WO 03/026744 and WO98/411711 teach drug delivery compositions for insertion into the anterior chamber of the eye comprising a viscoelastic and pharmaceuticals or medicaments. Listed among one of a large number of possible viscoelastic compositions is xanthan gum.

European Patent Application No. 0 974 320 A1 discloses a medical device for dispensing viscoelastic compositions characterized in that there are at least two discrete phases of a viscoelastic composition and each exhibiting a different viscosity. Xanthan gum was also listed among a large list of possible viscoelastic compositions.

While significant improvements have been made in the Theological properties of viscoelastic compositions, there still exists a need for a composition that has a relatively acceptable shape maintaining characteristics and a high pseudoplasticity index. The present invention addresses these and other needs.

### SUMMARY OF THE INVENTION

The present invention is a novel viscoelastic composition that has improved viscoelastic properties. The composition comprises an aqueous vehicle and a viscosurgically pure xanthan gum. In one embodiment, the viscoelastic composition has a minimum xanthan gum concentration of about 0.01% w/v and a maximum xanthan gum concentration of about 20% w/v based upon the total weight of the composition.

Particularly, the viscoelastic compositions have good shape maintaining characteristics at low-shear viscosity and a relatively high pseudoplasticity index. In another embodiment of the present invention, there is a method of temporarily maintaining space in a cavity in mammalian tissue. The method comprises injecting a viscoelastic composition comprising xanthan gum and an aqueous carrier into the cavity. At least a portion of the viscoelastic composition is removed from the cavity. The xanthan gum is viscosurgically pure.

In yet another embodiment, there is a method of protecting tissue from trauma during a surgical procedure, the method comprises coating at least a portion of the tissue with a viscoelastic composition comprising an aqueous vehicle and xanthan gum. After the tissue is coated, a surgical procedure is performed near the tissue. At least a portion of the viscoelastic composition is removed from the tissue after surgical procedure is performed. In one embodiment, at least a portion of the tissue in an anterior chamber of an eye is covered during the coating step. In another application, at least a portion of the corneal endothelium of an eye is coated.

In still another embodiment, there is a package for a viscoelastic composition. The package comprises a syringe containing a viscoelastic composition comprising an aqueous vehicle and xanthan gum. Optionally, the syringe has an outlet port. The package further comprises a cannula configured to sealably connect to the outlet port having a maximum inner diameter of about 1000 microns.

In another embodiment, there is an intraocular lens that comprises an intraocular lens coated with a viscoelastic composition according to one of the embodiments disclosed herein. The intraocular lens has improved properties for insertion into the vitreous of a patient.

In one embodiment, there is a method of replacing a natural lens from an eye. The method comprises the steps of:
(a) providing a passage through a sclera or cornea into an anterior chamber of the eye;
(b) removing at least a portion of the aqueous humor from the anterior chamber;
(c) inserting a viscoelastic composition into the anterior chamber, the viscoelastic composition comprises an aqueous vehicle and xanthan gum;
(d) phacoemulsifying a lens in the capsular bag of the eye;
(e) removing substantially all of the lens from the capsular bag;
(f) injecting the viscoelastic composition into the capsular bag; and
(g) inserting an intraocular lens into the capsular bag.

In one embodiment, there is an additional step of removing at least a portion of the viscoelastic composition from the capsular bag after the intraocular lens is inserted into the capsular bag. Optionally and additionally, at least a portion, and preferably substantially all, of the viscoelastic composition is removed from the anterior chamber. The phrase, “substantially all” as it relates to removing lenses and lens fragments, means a sufficient quantity to facilitate an effective removal of the lens. According to one embodiment, an effective removal of the lens requires a minimum of about 90% w/v, about 95% w/v, about 98% w/v or about 99% w/v of the lens. Typically, the method further includes a step of suturing the sclera after the intraocular lens is inserted into the capsular bag.

In an embodiment, there is a device for inserting an intraocular lens into a patient. The device comprises a loadable chamber and a tapered passage (and/or any other lens insertion device disclosed herein) wherein one of the loadable chamber and tapered passage is coated at least in part with a viscoelastic composition according to one or more embodiments disclosed herein.

In one embodiment, there is a method of inserting an intraocular lens into a capsular bag of an eye. The method comprises the steps of:
providing an eye with a cornea removed from the capsular bag and a passage into the capsular bag;
providing a lens insertion device comprising a loadable chamber configured to receive the intraocular lens, a tapered conduit having a first end connected to the loadable chamber and a second end, the second end is configured to penetrate into the passage, and a slidable actuator configured to actuate the intraocular lens through the conduit past the second end;
coating at least a portion of the intraocular lens with a viscoelastic composition comprising an aqueous vehicle and xanthan gum;
loading the intraocular lens into the loadable chamber;
inserting the conduit into the passage;
positioning the second end inside the capsular bag;
actuating the coated intraocular lens through the conduit into the capsular bag; and
removing the conduit from the passage.

In one application, the step of coating occurs after the step of loading. Additionally and optionally, the second end of the tapered conduit has an inner diameter that is a maximum of about 5 mm. Preferably the second end of the tapered conduit has an inner diameter that is a maximum of about 4 mm, about 3.5 mm, about 3 mm or about 2.8 mm.

### BRIEF DESCRIPTION OF THE DRAWINGS

FIG. 1 is a flow diagram of a process for purifying xanthan gum.

FIG. 2 is a graph plotting steady shear properties of a xanthan gum formulation and a hyaluronic acid formulation.

FIG. 3 is a graph comparing the dynamic oscillation data for xanthan gum and a comparative sample.

### DETAILED DESCRIPTION OF THE INVENTION

The present invention is a novel viscoelastic composition that has improved viscoelastic properties. The composition comprises an aqueous vehicle and a viscosurgically pure xanthan gum. The viscoelastic composition of one embodiment has a minimum xanthan gum concentration of about 0.01% w/v and a maximum xanthan gum concentration of about 20% w/v based upon the total weight of the composition. Particularly, the viscoelastic compositions of at least one embodiment of the present invention is capable of maintaining a zero-shear viscosity profile at higher shear rates relative to other leading viscoelastic compositions. Furthermore, the viscoelastic compositions of at least one embodiment of the present invention have a damping ratio that is higher than other viscoelastic compositions for viscosurgical applications.

For the purpose of this application, a viscoelastic composition has relatively viscous properties under low-shear and relatively elastic properties under high-shear conditions.

Viscosurgically pure as it pertains to this application refers to a viscoelastic composition or ingredient thereof that is sufficiently pure and free of impurities to meet or exceed the United States Food and Drug Administration standards for a viscosurgical viscoelastic effective at the time this application is effective.

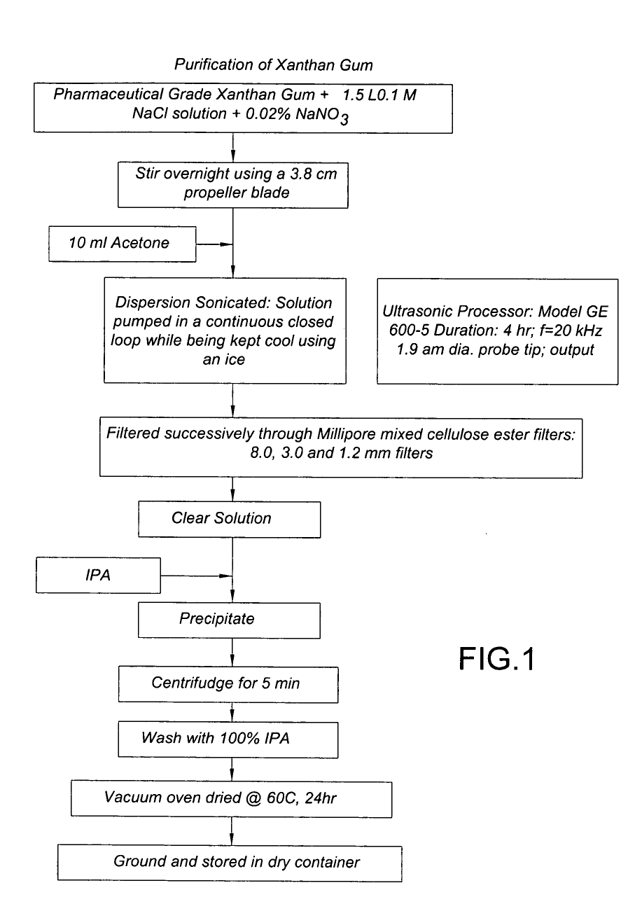

Xanthan Gum is defined as a hetero-polysaccharide that has main chain of glucose units that is substituted with trisacharide side chains comprising alpha-D-mannose with an acetyl group, beta-D-glucuronic acid and beta-D-mannose unit linked with a pyruvate group. Pharmaceutical/food grade Xanthan gum is commercially available under the trademark Vanzan® (including Vanzan® (NF-ED grade) from R.T.Vanderbilt Company, Inc., Norwalk, Conn. Xanthan gum can be clarified by purification according to the following process that is illustrated by way of example and not by limitation in a flow diagram of FIG. 1.

Pharmaceutical grade xanthan gum is mixed with a halide salt of sodium and/or potassium, preferably a chloride salt of sodium and/or potassium—most preferably sodium chloride. The resulting solution has a minimum of about 1 g/L, about 2 g/L, about 4 g/L or about 6 g/L of xanthan gum and/or a maximum of about 30 g/L, about 20 g/L, about 15 g/L, about 10 g/L or about 8 g/L of xanthan gum. In one embodiment, the concentration is about 6.5 g/L of xanthan gum. The concentration of the salt solution is in one embodiment a minimum of about 0.01 M, about 0.05M or about 0.1M and/or a maximum of about 0.5M, about 0.2 M or about 0.1 M. Optionally, an additional preservative is added to the solution in a preservative effective amount. Preservatives for ophthalmic solutions are generally known in the art.

Typically, the resulting dispersion is mixed overnight. Optionally, a free radical scavenger is added to the viscoelastic solution and the viscoelastic solution is sonicated for a minimum of about 1 hour and a maximum of about 20 hours (preferably 4 hours) in an ice bath using an ultrasonic processor to disrupt cell walls and separate organelle material from the polysaccharide. One example of a free radical scavenger is acetone. By way of example, the solution is maintained in an ice bath during sonication and is constantly being recirculated using, for example, a peristaltic pump.

After sonication, the resulting solution is pressure filtered in one or more stages to remove any particulate that is greater than about 1 micron. Typically, the filter with the smallest pore size has a minimum pore size of about 0.001, about 0.01, about 0.1 microns or about 1 micron and/or a maximum pore size of about 5.0 microns, about 3.0 microns, about 2.0 microns or about 1.0 microns. In one embodiment, the resulting solution is filtered with an 8 micron filter, followed by a 3 micron filter, followed by a 1.2 micron filter. The filtration yields an optically clear solution. In one embodiment, the purification process yields a xanthan gum solution that has a maximum of about 500 ppm endotoxins. Preferably, the xanthan gum has a maximum of about 100 ppm, about 50 ppm, about 10 ppm, about 5 ppm or about 1 ppm of endotoxins. Most preferably, the xanthan gum has undetectable levels of endotoxins or no endotoxins.

Xanthan gum is then precipitated from the solution by adding a C₁₋₄ alcohol, preferably isopropyl alcohol. The precipitate is washed again with IPA and dried. In one embodiment the solution is dried for a minimum of 6 hours, 8 hours, 10 hours or 12 hours at a temperature that is a minimum of about 40 C, about 50 C, or about 60 C and a maximum of about 120 C, about 100 C, or about 80 C in a vacuum oven. The dried polymer is then ground and stored or alternatively reconstituted into a viscoelastic formula according to the present invention.

The average molecular weight of the xanthan gum is a minimum of about 900 kD and a maximum of about 50,000 kD. Preferably, the average molecular weight is a minimum of about 1,000 kD, about 2,000 kD, about 5,000 kD, or about 10,000 kD and/or is a maximum of about 40,000 kD, about 30,000 kD, or about 20,000 kD.

As noted the viscoelastic composition has a minimum xanthan gum concentration of about 0.05% w/v and a maximum xanthan gum concentration of about 9% w/v, based upon the total weight of the composition. Typically, the minimum xanthan gum concentration is about 1% w/v, about 1.5% w/v, about 2% w/v, about 3% w/v or about 4% w/v based upon the total weight of the viscoelastic composition. Typically, the maximum xanthan gum concentration is about 10% w/v, about 8% w/v, about 6% w/v, about 4% w/v, about 3% w/v or about 2% w/v based upon the total weight of the viscoelastic composition. Preferably the xanthan gum concentration is a minimum of about 1% w/v and a maximum of about 3% w/v.

Optionally, the pH is adjusted to a desired range having a minimum of about 7 and a maximum of about 8. In one embodiment, the pH of the viscoelastic composition is a minimum of about 7.1, about 7.2 or about 7.3 and a maximum of about 7.8, about 7.6, about 7.4 or about 7.3. The pH is adjusted with physiological acids or bases such as acetic acid, acetate, carbonic acid, carbonate, phosphoric acid, phosphate. After the pH is adjusted, the pH is typically maintained with a buffer system.

Preferably, a buffer system does not substantially affect the viscoelastic properties of the viscoelastic composition. Desirably, the buffer system does not cause irritation at the amounts used in the viscoelastic composition. Buffer systems useful in the present invention include but are not limited to a N-2hydroxyethylpiperazine-N′-ethane sulphonic acid (HEPES) buffer system, a carbonate buffer system, and a phosphate buffer system—more preferably a phosphate buffered saline (PBS) system.

In one embodiment, the osmolality of the composition is a minimum of about 200 mOsmol/L and a maximum of about 400 mOsmol/L. Typically, the osmolality of the viscoelastic composition is a minimum of about 220 mOsmol/L, about 260 mOsmol/L, about 280 mOsmol/L, about 300 mOsmol/L or about 320 mOsmol/L. Typically, the osmolality of the viscoelastic composition is a maximum of about 400 mOsmol/L, about 380 mOsmol/L, about 360 mOsmol/L or about 340 mOsmol/L. Most preferably, the osmolality of the viscoelastic composition is about 340 mOsmol/L.

In one embodiment, the osmolality is altered by adding an osmolality-adjusting agent that is known in the art. Typically, osmolality-adjusting agents are capable of increasing the osmolality of the viscoelastic composition without causing irritation of the eye at the quantity needed to appropriately adjust the osmolality. Suitable osmolality-adjusting agents include but are not limited to glycerin. Most preferably, the osmolality-adjusting agent is added in an amount that is a minimum of about 0.1% w/v, about 1% w/v or about 1.5% w/v and a maximum of about 5% w/v, about 2.5% w/v or about 2% w/v.

The viscoelastic properties of the viscoelastic composition of the present invention are important to their effectiveness in the surgical procedure. Zero-shear viscosity is a good indicator of how a viscoelastic composition will maintain the space of a cavity in human tissue. Zero-shear viscosity is the extrapolation of the viscosity of a liquid to a zero-shear rate from measurements of viscosity as the shear rate approaches zero measured on a plate and cone rheometer at 37° C.

In one embodiment, the viscoelastic composition has a zero-shear viscosity that is a minimum of about 100 Pa-s and a maximum of about 5,000 Pa-s. Preferably, the zero-shear viscosity of the composition is a minimum of about 200 Pa-s, about 300 Pa-s, about 400 Pa-s, about 500 Pa-s or about 600 Pa-s and/or a maximum of about 4,000 Pa-s, about 3,000 Pa-s, about 2,000 Pa-s, or about 1,000 Pa-s.

High-shear conditions refer to shear conditions having a minimum shear force of about 100 sec-1. High-shear viscosity, for the purpose this patent application, is the viscosity of a liquid measured on a plate and cone rheometer at 37° C. with a shear rate of 1000 sec-1. In one embodiment, the high-shear viscosity of the viscoelastic composition is a minimum of about 0.01 Pa-s and a maximum of about 30 Pa-s. Preferably the high-shear viscosity of the composition is a minimum of about 0.03 Pa-s, about 0.05 Pa-s, about 0.07 Pa-s or about 0.1 Pas and/or a maximum of about 20 Pa-s, about 10 Pa-s, about 5 Pa-s or about 1 Pa-s.

The pseudoplasticity index is another important factor. The pseudoplasticity measures the degree of change in viscosity from a low shear state to a high shear state. For the purpose of this application, pseudoplasticity is defined as the ratio of viscosity at a shear rate of 0.3 s-1 to the viscosity at a shear rate of 300 s-1. In one embodiment, the pseudoplasticity index of the viscoelastic composition is a minimum of about 100. Preferably the pseudoplasticity index of the viscoelastic composition is a minimum of about 400, about 600, about 800, about 1000 or about 5000.

In one embodiment, there is a method of replacing a natural lens from an eye. Examples of procedures for removing a lens from a patient's eye include but are not limited to U.S. Pat. No. 3,589,363 (cataract surgery), U.S. Pat. No. 3,693,613 (phacoemulsification) and U.S. Pat. No. 5,718,676 (process using micro flow needle), which are all incorporated herein by reference in their entirety. The process generally includes providing a passage through a sclera or cornea into an anterior chamber of the eye. The process involves making a small incision into the sclera or cornea. Alternatively or additionally, a cannula or trochar is used to create a passage through the sclera or cornea into the capsular bag. Preferably, the incision or passage is as small as possible. Preferably the incision or passage is smaller than about 5 mm, about 4 mm or about 3 mm. Thereafter, the aqueous humor is withdrawn or otherwise removed from the anterior chamber of the eye.

A viscoelastic composition, according to any one of the embodiments or combinations, is inserted into the anterior chamber. The viscoelastic, of one embodiment, maintains the space in the anterior chamber. The viscoelastic of one embodiment, coats the tissue in the wall of the anterior chamber.

According to one embodiment, there is a device for delivering a viscoelastic composition into the anterior chamber of a patient's eye. Alternatively, there is a package for viscoelastic composition. The package or device comprises a syringe containing a viscoelastic composition comprising an aqueous vehicle and xanthan gum. In one embodiment, the syringe has an outlet port, the package further comprising a cannula configured to sealably connect to the outlet port having a maximum inner diameter of about 1000 microns. Typically, the maximum inner diameter is about 700 microns, about 500 microns or about 300 microns.

Once the viscoelastic composition is inserted into the anterior chamber the corneal lens is removed. The technique for removing the lens includes performing a capsulorhexis incision and breaking down the lens into smaller pieces through phacoemulsification or other known techniques. Thereafter, the pieces are removed by aspiration.

The viscoelastic composition is inserted into the capsular bag for space maintenance purposes. Moreover, the viscoelastic composition coats the capsular bag and protects it for additional steps in the surgical procedure.

According to one embodiment, the intraocular lens is inserted into the capsular bag. Typically, there is a method of inserting an intraocular lens into a capsular bag of an eye. The method comprises providing a lens insertion device comprising a loadable chamber configured to receive the intraocular lens, a tapered conduit having a first end connected to the loadable chamber and a second end. The second end is configured to penetrate through the passage in the corneal lens and into the capsular bag. An example of a lens insertion device is found in U.S. Pat. No. 6,558,419, which is incorporated herein by reference in its entirety. The lens insertion device is further configured with a slidable actuator. The slidable actuator of one embodiment is configured to actuate the intraocular lens through the conduit past the second end. Typically, the second end of the tapered conduit has an inner diameter that is a maximum of about 5 mm. Preferably the second end of the tapered conduit has an inner diameter that is a maximum of about 4 mm about 3.5 mm, about 3 mm or about 2.8 mm.

Prior to deployment, at least a portion of the intraocular lens is coated with a viscoelastic composition according to any one of the embodiments of the present invention. The intraocular lens is loaded into the loadable chamber either before or after it is coated. The conduit is inserted through the passage. In one embodiment, the conduit is coated with a viscoelastic composition according to the present invention. The actuator forces the intraocular lens through the passage and into the capsular bag. After the intraocular lens is deployed, the conduit is removed from the passage. Thus in one embodiment, there is a coated intraocular lens wherein the coating comprises a viscoelastic composition according to any one or more embodiment disclosed herein.

Typically, at least a portion of the viscoelastic composition is removed from the capsular bag and/or anterior chamber. A physiological solution is then used to fill the anterior chamber. The sclera and/or cornea are sutured to close the passage.

In one embodiment, of the present invention, one or more viscoelastic compositions set forth in the present invention are used to maintain the space of a cavity in a patient's tissue. The process includes injecting a viscoelastic composition comprising xanthan gum and an aqueous carrier into the cavity. After the cavity is maintained for a period of time, at least a portion of the viscoelastic composition is removed from the cavity. The space is often maintained during a surgical procedure that often occurs in the cavity itself. In one embodiment, the surgery occurs in the patient's eye. In another embodiment, the surgical procedure is cataract removal. The cavity is the anterior chamber of the patient's eye and/or the capsular bag of the patient's eye.

The use of xanthan gum in surgery also protects tissue from damage during the surgical procedure. The viscoelastic composition coats the surface of the tissue. A surgical procedure is performed near the tissue. The viscoelastic composition cushions the tissue from physical trauma. Preferably, the viscoelastic has dispersive viscoelastic properties to protect the tissue. In one embodiment, the process of coating covers at least a portion of the tissue in an anterior chamber of an eye. In another embodiment, the step of coating covers at least a portion of the corneal endothelium of an eye. The surgical procedure further includes removing at least a portion of the viscoelastic composition from the tissue.

In another embodiment, there is an intraocular lens that comprises an intraocular lens coated with a viscoelastic composition according to one of the embodiments disclosed herein. The intraocular lens has improved properties for insertion into the vitreous of a patient.

In an embodiment, there is a device for inserting an intraocular lens into a patient. The device comprises a loadable chamber and a tapered passage (and/or any other lens insertion device disclosed herein) wherein one of the loadable chamber and tapered passage is coated at least in part with a viscoelastic composition according to one or more embodiments disclosed herein.

### EXAMPLE 1

### Purification of Xanthan Gum

Semi-dilute solutions (c>1 g/100 ml) of commercially available Xanthan Gum are opaque in appearance. The following process was utilized to obtain an optically clear solution of xanthan gum. Ten g. of a pharmaceutical grade of Xanthan Gum (Vanzan NF-ED from R.T.Vanderbilt Co. Lot # 1302, Exp.: March 2004) was added to 1.5 L 0.1M NaCl solution along with a preservative. The dispersion was mixed overnight and sonicated using an ultrasonic processor to disrupt cell walls and separate organelle material from the polysaccharide. Acetone was added as a free radical scavenger to quench the free radicals generated during sonication and thus minimizing chain scission. During sonication, the solution was maintained in an ice bath while constantly being recirculated using a peristaltic pump. The resulting solution was successively pressure filtered with air through 8, 3 and 1.2 um filter membranes. Optically clear solution was thus obtained. Xanthan gum was precipitated using isopropyl alcohol. The precipitate was washed again with IPA and dried overnight at 60 C in a vacuum oven. The dried polymer was then ground and stored.

### EXAMPLE 2

### Preparation of Samples

The following samples were prepared or obtained for testing:

Solution 1 is a hyaluronic acid viscoelastic sold under the brand name Amvisc Plus by Bausch & Lomb, Rochester, N.Y., Lot # B010420.

Solution 2 is prepared by dissolving 15 mg of xanthan gum that was purified according to the procedure of Example 1 in 0.1M NaCl solution resulting in an optically clear viscoelastic solution.

### EXAMPLE 3

### Steady State Shear Test

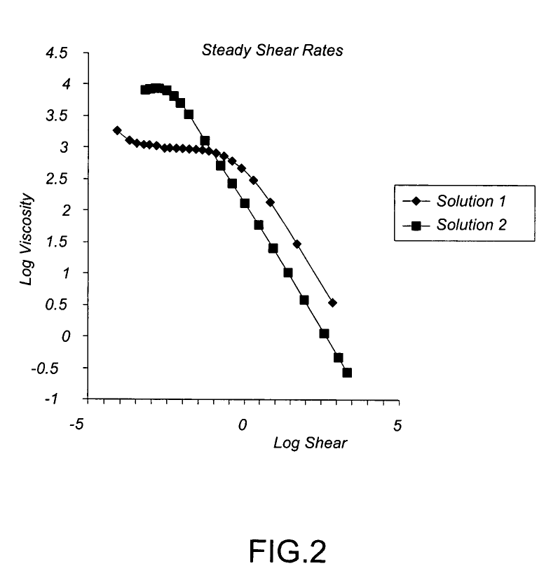

A TA Instruments T-1000R rheometer with a 50-mm diameter cone-and-plate (2 degrees) geometry was used to perform rheological tests on Solution 1 (hyaluronic acid) and Solution 2 (xanthan gum). The geometry gap used was 48 um. Steady Shear experiments were conducted using torque as the control parameter. The steady shear test was carried out at room temperature. Results of the steady state shear test for Solution 1 is shown in Table 1 below. The results of the steady shear test for Solution 2 is shown in Table 2 below. FIG. 2 compares the results of the steady shear viscosity test for Solution 1 and Solution 2.

TABLE 1
Steady Shear Viscosity Test
Solution 1: Hyaluronic Acid

| Shear Rate | Viscosity | Log shear rate | Log viscosity |
|---|---|---|---|
| (1/sec) | (Poise) | (log 1/sec) | (log poise) |
| 8.1 × 10⁻⁵ | 1850 | −4.09 | 3.27 |
| 1.9 × 10⁻⁴ | 1240 | −3.72 | 3.09 |
| 3.3 × 10⁻⁴ | 1160 | −3.49 | 3.06 |
| 5.5 × 10⁻⁴ | 1080 | −3.26 | 3.03 |
| 8.6 × 10⁻⁴ | 1100 | −3.07 | 3.04 |
| 1.4 × 10⁻³ | 1050 | −2.85 | 3.02 |
| 2.5 × 10⁻³ | 939 | −2.60 | 2.97 |
| 3.8 × 10⁻³ | 1000 | −2.42 | 3.00 |
| 6.3 × 10⁻³ | 950 | −2.20 | 2.98 |
| 0.010 | 950 | −2.00 | 2.98 |
| 0.016 | 940 | −1.81 | 2.97 |
| 0.026 | 930 | −1.60 | 2.97 |
| 0.041 | 909 | −1.38 | 2.96 |
| 0.069 | 871 | −1.16 | 2.94 |
| 0.18 | 812 | −0.934 | 2.91 |
| 0.21 | 724 | −0.684 | 2.86 |
| 0.39 | 608 | −0.408 | 2.78 |
| 0.81 | 465 | −0.0911 | 2.67 |
| 2.0 | 297 | 0.303 | 2.47 |
| 7.0 | 135 | 0.847 | 2.13 |
| 52 | 28.9 | 1.71 | 1.46 |
| 72 | 3.29 | 2.86 | 0.517 |

TABLE 2
Steady Shear Viscosity Test
Solution 2: Xanthan Gum

| Shear Rate | Viscosity | Log Shear Rate | Log Viscosity |
|---|---|---|---|
| (1/sec) | (Poise) | (log 1/sec) | (Log Poise) |
| 6.53 × 10⁻⁴ | 8050 | −3.18 | 3.91 |
| 7.63 × 10⁻⁴ | 8670 | −3.12 | 3.94 |
| 9.48 × 10⁻⁴ | 8790 | −3.02 | 3.94 |
| 1.27 × 10⁻³ | 8240 | −2.90 | 3.92 |
| 1.47 × 10⁻³ | 9000 | −2.83 | 3.95 |
| 1.84 × 10⁻³ | 9040 | −2.74 | 3.96 |
| 2.60 × 10⁻³ | 8050 | −2.58 | 3.91 |
| 3.34 × 10⁻³ | 7880 | −2.48 | 3.90 |
| 5.16 × 10⁻³ | 6430 | −2.29 | 3.81 |
| 8.37 × 10⁻³ | 4990 | −2.08 | 3.70 |
| 0.0160 | 3280 | −1.80 | 3.52 |
| 0.0523 | 1270 | −1.28 | 3.10 |
| 0.161 | 518 | −0.793 | 2.71 |
| 0.402 | 261 | −0.395 | 2.41 |
| 1.04 | 127 | 0.0166 | 2.10 |
| 2.86 | 58.1 | 0.457 | 1.76 |
| 8.54 | 24.5 | 0.931 | 1.39 |
| 26.2 | 10.0 | 1.42 | 1.00 |
| 89.3 | 3.70 | 1.95 | 0.568 |
| 379 | 1.09 | 2.58 | 0.0358 |
| 1130 | 0.448 | 3.05 | −0.348 |
| 2330 | 0.265 | 3.37 | −0.577 |

Solution 1 (hyaluronic acid) as illustrated in FIG. 2. Solution 2 had a higher zero shear viscosity (h0) but shear thinned more readily, at lower shear rates, than Solution 1. Thus, Solution 2 (xanthan gum) exhibits more pseudoplasticity whereas Solution 1 (hyaluronic acid) shows a more Newtonian behavior at low shear rates. While Solution 2 (xanthan gum) has more pseudoplasticity than Solution 1 (hyaluronic acid) at lower shear rates, Solution 2 (xanthan gum) has higher viscosity than solution 1 (hyaluronic acid) until a shear rate of about 10 rad/sec.

### EXAMPLE 4

### Dynamic Oscillation

A TA Instruments T-1000R rheometer with a 50-mm diameter cone-and-plate geometry was used to perform Rheological tests on the above two solutions. The geometry gap used was 48 um. Steady Shear experiments were conducted using torque as the control parameter and Dynamic Oscillation tests were carried out at 1% strain control. Both tests were carried out at room temperature.

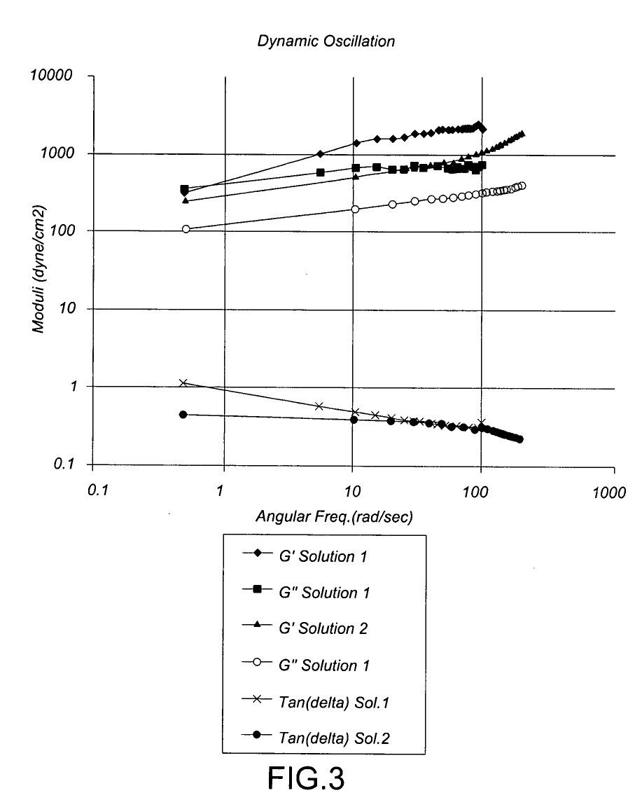

FIG. 3 shows a comparison of the dynamic storage and loss modulus as a function of angular frequency for Solution 1 (hyaluronic acid) and Solution 2 (xanthan gum). The elastic and loss moduli cross over at about 1 rad/sec for Solution 1 whereas, for Solution 2, these values could not be measured. Both Solution 1 and Solution 2 exhibit characteristics of a concentrated polymer solution. Solution 1 tends to become rubber like at higher frequencies as suggested by the elastic modulus plateau. Solution 2 behaves more like an elastic solid at higher frequencies as indicated by the rising elastic modulus. Also, the elastic modulus of Solution 2 is consistently greater than its loss modulus over the frequency sweep.

The high zero shear viscosity of Solution 2 is likely to provide very good capsular space maintenance during IOL implantation similar to or better than other cohesive viscoelastics. But, due to its higher degree of pseudoplasticity, Solution 2 can be injected more easily using a fine cannula relative to other cohesive viscoelastics. Also, Solution 2 is likely to disperse and coat the endothelial tissues under the slightest agitation providing better protection than Solution 1. Thus, a viscoelastic solution comprising xanthan gum could potentially provide an excellent combination of both cohesive and dispersive properties, which are difficult to obtain from any of the commercially available viscoelastics.

What is claimed is:

1. A composition comprising an aqueous vehicle and a viscosurgically pure xanthan gum.

2. The composition of claim 1, wherein the xanthan gum concentration is a minimum of about 0.01% w/v and a maximum of about 20% w/v based upon the total weight of the composition.

3. The composition of claim 1, wherein the average molecular weight of the xanthan gum is a minimum of about 900 kD and a maximum of about 50,000 kD.

4. The composition of claim 1, wherein the composition has a minimum xanthan gum concentration of about 0.05% w/v and a maximum xanthan gum concentration of about 9% w/v, based upon the total weight of the composition.

5. The composition of claim 1, wherein the osmolality of the composition is a minimum of about 200 mOsmol/L and a maximum of about 400 mOsmol/L.

6. The composition of claim 1, wherein the zero-shear viscosity of the composition is a minimum of about 100 Pa-s and a maximum of about 5,000 Pa-s.

7. The composition of claim 1, wherein the high-shear viscosity of the composition is a minimum of about 0.01 Pa-s and a maximum of about 30 Pa-s.

8. The composition of claim 1, wherein the pseudoplasticity index of the viscoelastic composition is a minimum of about 100.

9. The composition of claim 1, wherein the pH of the composition is a minimum of about 7 and a maximum of about 8.

10. The composition of claim 1, wherein the xanthan gum has a maximum of about 500 ppm endotoxins.

11. A method of temporarily maintaining space in a cavity in mammalian tissue, the method comprising the steps of:
(a) injecting a viscoelastic composition comprising xanthan gum and an aqueous carrier into the cavity; and
(b) removing at least a portion of the viscoelastic composition from the cavity.

12. The method of claim 11, wherein the xanthan gum is derived from a microbial fermentation process.

13. The method of claim 11, wherein the xanthan gum is purified to remove particulate having with a filter having a minimum pore size of about 0.001 microns and a maximum of about 5.0 microns.

14. The method of claim 11, wherein the xanthan gum is a viscosurgically pure xanthan gum.

15. The method of claim 11, wherein the xanthan gum has a maximum of about 500 ppm endotoxins.

16. The method of claim 11, wherein the xanthan gum concentration is a minimum of about 0.01% w/v and a maximum of about 20% w/v based upon the total weight of the composition.

17. The method of claim 11, wherein the average molecular weight of the xanthan gum is a minimum of about 900 kD and a maximum of about 50,000 kD.

18. The method of claim 11, wherein the composition has a minimum xanthan gum concentration of about 0.05% w/v and a maximum xanthan gum concentration of about 9% w/v, based upon the total weight of the composition.

19. The method of claim 11, wherein the osmolality of the composition is a minimum of about 200 mOsmol/L and a maximum of about 400 mOsmol/L.

20. The method of claim 11, wherein the zero-shear viscosity of the composition is a minimum of about 100 Pa-s and a maximum of about 5,000 Pa-s.

21. The method of claim 11, wherein the high-shear viscosity of the composition is a minimum of about 0.01 Pa-s and a maximum of about 30 Pa-s.

22. The method of claim 11, wherein the pseudoplasticity index of the viscoelastic composition is a minimum of about 100.

23. The method of claim 11, wherein the pH of the composition is a minimum of about 7 and a maximum of about 8.

24. A method of protecting tissue from trauma during a surgical procedure, the method comprising the steps of:
(a) coating at least a portion of the tissue with a viscoelastic composition comprising an aqueous vehicle and xanthan gum;
(b) performing a surgical procedure near the tissue after the step (a) coating; and
(c) removing at least a portion of the viscoelastic composition from the tissue before the step (b) performing.

25. The method of claim 24, wherein the step (a) coating covers at least a portion of the tissue in an anterior chamber of an eye.

26. The method of claim 24, wherein the step (a) coating covers at least a portion of the corneal endothelium of an eye.

27. The method of claim 24, wherein the xanthan gum is derived from a microbial fermentation process.

28. The method of claim 24, wherein the xanthan gum is purified to remove particulate having with a filter having a minimum pore size of about 0.001 microns and a maximum of about 5.0 microns.

29. The method of claim 24, wherein the xanthan gum is a viscosurgically pure xanthan gum.

30. The method of claim 24, wherein the xanthan gum has a maximum of about 500 ppm endotoxins.

31. The method of claim 24, wherein the xanthan gum concentration is a minimum of about 0.01% w/v and a maximum of about 20% w/v based upon the total weight of the composition.

32. The method of claim 24, wherein the average molecular weight of the xanthan gum is a minimum of about 900 kD and a maximum of about 50,000 kD.

33. The method of claim 24, wherein the composition has a minimum xanthan gum concentration of about 0.05% w/v and a maximum xanthan gum concentration of about 9% w/v, based upon the total weight of the composition.

34. The method of claim 24, wherein the osmolality of the composition is a minimum of about 200 mOsmol/L and a maximum of about 400 mOsmol/L.

35. The method of claim 24, wherein the zero-shear viscosity of the composition is a minimum of about 100 Pa-s and a maximum of about 5,000 Pa-s.

36. The method of claim 24, wherein the high-shear viscosity of the composition is a minimum of about 0.01 Pa-s and a maximum of about 30 Pa-s.

37. The method of claim 24, wherein the pseudoplasticity index of the viscoelastic composition is a minimum of about 100.

38. The method of claim 24, wherein the pH of the composition is a minimum of about 7 and a maximum of about 8.

39. A package for a viscoelastic composition, the package comprising a syringe containing a viscoelastic composition comprising an aqueous vehicle and xanthan gum.

40. The package of claim 39, wherein the syringe has an outlet port, the package further comprising a cannula configured to sealably connect to the outlet port having a maximum inner diameter of about 1000 microns.

41. The package of claim 39, wherein the xanthan gum is derived from a microbial fermentation process.

42. The package of claim 39, wherein the xanthan gum is purified to remove particulate having with a filter having a minimum pore size of about 0.001 microns and a maximum of about 5.0 microns.

43. The package of claim 39, wherein the xanthan gum is a viscosurgically pure xanthan gum.

44. The method of claim 39, wherein the xanthan gum has a maximum of about 500 ppm endotoxins.

45. A method of replacing a natural lens from an eye, the method comprising the steps of:
(a) providing a passage through a sclera or cornea into an anterior chamber of the eye;
(b) removing at least a portion of the aqueous humor from the anterior chamber;
(c) inserting a viscoelastic composition into the anterior chamber, the viscoelastic composition comprises an aqueous vehicle and xanthan gum;
(d) phacoemulsifying a lens in the capsular bag of the eye;
(e) removing substantially all of the lens from the capsular bag;
(f) injecting the viscoelastic composition into the capsular bag; and
(g) inserting an intraocular lens into the capsular bag.

46. The method of claim 45, further comprising the step of removing at least a portion of the viscoelastic composition from the capsular bag.

47. The method of claim 45, further comprising the step of removing at least a portion of the viscoelastic composition from the anterior chamber.

48. The method of claim 45, further comprising the step of suturing the sclera after the step (g) inserting an intraocular lens.

49. The method of claim 45, wherein the xanthan gum is a viscosurgically pure xanthan gum.

50. The method of claim 45, wherein step (d) removing further comprises removing the lens by a procedure selected from the group consisting of extracapsular cataract extraction and phacoemulsification.

51. The method of claim 45, wherein the osmolality of the viscoelastic composition is a minimum of about 200 mOsmol/L and a maximum of about 400 mOsmol/L.

52. The method of claim 45, wherein the zero-shear viscosity of the viscoelastic composition is a minimum of about 10 Pa-s and a maximum of about 300 Pa-s.

53. The method of claim 45, wherein the high-shear viscosity of the viscoelastic composition is a minimum of about 0.1 Pa-s and a maximum of about 30 Pa-s.

54. The method of claim 45, wherein the viscoelastic composition has a pseudoplasticity index that is a minimum of about 100.

55. The method of claim 45, wherein the xanthan gum comprises b-D-mannuronic acid and a-L-guluronic acid, wherein the ratio of the b-D-mannuronic acid to a-L-guluronic acid is in a range having a minimum of about 1 and a maximum of about 4.

56. A method of inserting an intraocular lens into a capsular bag of an eye, the method comprising the steps of:
providing an eye with a cornea removed from the capsular bag and a into the capsular bag;
providing a lens insertion device comprising a loadable chamber configured to receive the intraocular lens, a tapered conduit having a first end connected to the loadable chamber and a second end, the second end is configured to penetrate into the passage, and a slidable actuator configured to actuate the intraocular lens through the conduit past the second end;
coating at least a portion of the intraocular lens with a viscoelastic composition comprising an aqueous vehicle and xanthan gum;
loading the intraocular lens into the loadable chamber;
inserting the conduit into the passage;
positioning the second end inside the capsular bag;
actuating the coated intraocular lens through the conduit into the capsular bag; and
removing the conduit from the passage.

57. The method of claim 54, wherein the step of coating occurs after the step of loading.

58. The method of claim 54, wherein the second end of the tapered conduit has an inner diameter that is a maximum of about 5 mm.

59. The method of claim 54, wherein the xanthan gum is a viscosurgically pure xanthan gum.
